(19) United States
(12) Patent Application Publication
(10) Pub. No.: US 2022/0183570 A1
(43) Pub. Date: Jun. 16, 2022

(54) BLOOD FLOW MEASURING DEVICE

(71) Applicant: SONY GROUP CORPORATION, TOKYO (JP)

(72) Inventor: ATSUSHI ITO, TOKYO (JP)

(21) Appl. No.: 17/432,255

(22) PCT Filed: Feb. 6, 2020
(86) PCT No.: PCT/JP2020/004485

§ 371 Date: Aug. 19, 2021

(30) Foreign Application Priority Data
Mar. 1, 2019 (JP) 2019-037685

(51) Int. Cl. A61B 5/026; A61B 5/00

(52) U.S. Cl. CPC A61B 5/026; A61B 5/7225

(57) ABSTRACT

Provided is a blood flow measuring device that is provided with at least one AD conversion unit and processes a plurality of reflected light signals. Provided is a blood flow measuring device provided with at least one light source unit, a plurality of light reception units, a plurality of signal processing units, at least one multiplexing unit, and at least one AD conversion unit, in which the light source unit irradiates a living body with coherent light, each of the plurality of light reception units receives reflected light of the coherent light and converts intensity of the reflected light into a current signal, each of the plurality of signal processing units converts the current signal into a voltage signal, the multiplexing unit multiplexes a plurality of voltage signals into at least one multiplexed voltage signal, and the AD conversion unit samples the multiplexed voltage signal to obtain a multiplexed digital signal.

### TECHNICAL FIELD

The present technology relates to a blood flow measuring device.

### BACKGROUND ART

Conventionally, a technology of irradiating the skin of a living body with a laser beam and the like close to coherent light to measure a blood flow of the blood vessel near the skin surface in a non-invasive manner has been utilized. This technology utilizes the Doppler effect in which a frequency of a reflected light signal changes according to a moving speed of an object. It is known that a blood flow velocity correlating with an average frequency may be calculated by calculating the average frequency from frequency distribution of this reflected light signal.

However, since a sampling interval for calculating the average frequency is short, this reflected light signal data contains a lot of noise. Therefore, in order to obtain a low-noise signal, Patent Documents 1 and 2 disclose the technology of performing averaging processing on a plurality of reflected light signals obtained from a plurality of light reception units.

### CITATION LIST

### Patent Document

Patent Document 1: Japanese Patent Application Laid-Open No. 8-182658
Patent Document 2: U.S. Pat. No. 4,109,647

### SUMMARY OF THE INVENTION

### Problems to be Solved by the Invention

In order to perform averaging processing on a plurality of reflected light signals, an AD conversion unit that converts the reflected light signal being an analog signal into a digital signal is required for each light reception unit.

However, in a case where the AD conversion unit is installed for each light reception unit, there is a problem that a blood flow measuring device becomes a large and expensive device.

Therefore, a principal object of the present technology is to provide a blood flow measuring device that is provided with at least one AD conversion unit and processes a plurality of reflected light signals.

### Solutions to Problems

The present technology provides a blood flow measuring device provided with at least one light source unit, a plurality of light reception units, a plurality of signal processing units, at least one multiplexing unit, and at least one AD conversion unit, in which the light source unit irradiates a living body with coherent light, each of the plurality of light reception units receives reflected light of the coherent light and converts intensity of the reflected light into a current signal, each of the plurality of signal processing units converts the current signal into a voltage signal, the multiplexing unit multiplexes a plurality of voltage signals into at least one multiplexed voltage signal, and the AD conversion unit samples the multiplexed voltage signal to obtain a multiplexed digital signal.

The signal processing unit may include a smoothing unit.

The smoothing unit includes a charging switch, and a capacitor, the charging switch alternately repeats turning on and off, and the capacitor alternately repeats a state of being charged with an electric charge regarding the voltage signal and a state of discharging the electric charge, so that the electric charge may be sampled.

The blood flow measuring device is further provided with an averaging processing unit, and the averaging processing unit may perform averaging processing on the multiplexed digital signal to obtain a low-noise signal.

The blood flow measuring device is further provided with a timing control unit, and the timing control unit may synchronize a discharge timing of the capacitor with a sampling timing of the AD conversion unit.

The timing control unit may synchronize discharge timings of a plurality of capacitors.

Phases of a plurality of sampling timings may be different from each other.

Cycles of a plurality of sampling timings may be different from each other.

The smoothing unit is provided with a plurality of charging switches, a plurality of sampling switches, and a plurality of capacitors, and at a timing at which each of the plurality of charging switches is turned on or off, an electric charge regarding the voltage signal with which each of the plurality of capacitors is charged is discharged and each of the plurality of sampling switches is turned on or off, so that the electric charge may be sampled.

### BRIEF DESCRIPTION OF DRAWINGS

FIG. 1 is a configuration diagram of one embodiment of a blood flow measuring device according to the present technology.

FIG. 2 is a configuration diagram of one embodiment of the blood flow measuring device according to the present technology.

FIG. 3 is a configuration diagram of one embodiment of the blood flow measuring device according to the present technology.

FIG. 4 is a circuit diagram of one embodiment of the blood flow measuring device according to the present technology.

FIG. 5 is a configuration diagram of one embodiment of a multiplexing unit according to the present technology.

FIG. 6 is a circuit diagram of one embodiment of the blood flow measuring device according to the present technology.

FIG. 7 is an illustrative view illustrating a simulation result of a time constant and a noise variation coefficient.

FIG. 8 is a configuration diagram of one embodiment of the blood flow measuring device according to the present technology.

FIG. 9 is a timing chart of a charging switch and an AD conversion unit according to the present technology.

FIG. 10 is a configuration diagram of one embodiment of the blood flow measuring device according to the present technology.

FIG. 11 is an illustrative view illustrating a waveform of a voltage signal according to the present technology.

FIG. 12 is an illustrative view illustrating a waveform of a voltage signal according to the present technology.

FIG. 13 is a configuration diagram of one embodiment of the blood flow measuring device according to the present technology.

FIG. 14 is a timing chart of a charging switch and a sampling switch according to the present technology.

### MODE FOR CARRYING OUT THE INVENTION

Hereinafter, a preferred mode for carrying out the present technology is described with reference to the attached drawings. Note that, embodiments hereinafter described are representative embodiments of the present technology, and the scope of the present technology is not limited to them. Note that, the present technology is described in the following order.

1. First Embodiment according to Present Technology (Basic Configuration)

2. Second Embodiment according to Present Technology (Configuration of Plural Groups)

3. Third Embodiment of Present Technology (Configuration in Two-Dimensional Direction)

1. First Embodiment according to Present Technology (Basic Configuration)

(1) Overview

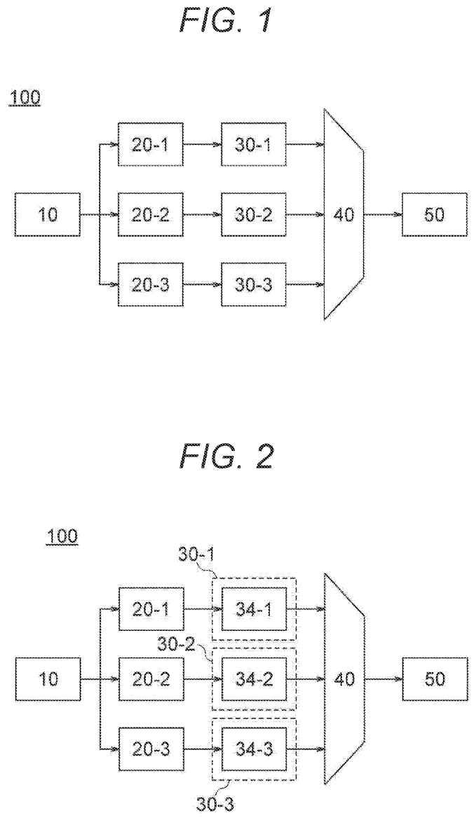

FIG. 1 is a configuration diagram of a blood flow measuring device according to a first embodiment of the present technology. As illustrated in FIG. 1, a blood flow measuring device 100 is provided with at least one light source unit 10, a plurality of light reception units (20-1, 20-2, 20-3), a plurality of signal processing units (30-1, 30-2, 30-3), at least one multiplexing unit 40, and at least one AD conversion unit 50. The light reception units (20-1, 20-2, 20-3), the signal processing units (30-1, 30-2, 30-3), the multiplexing unit 40, and the AD conversion unit 50 are electrically connected to one another.

Note that, the configuration diagram of one embodiment of the blood flow measuring device 100 in FIG. 1 includes, for example, three light reception units (20-1, 20-2, 20-3) and three signal processing units (30-1, 30-2, 30-3), but the number of a plurality of light reception units (20-1, 20-2, 20-3) and that of a plurality of signal processing units (30-1, 30-2, 30-3) are not limited to three. It is desirable that the number of the light reception units and the signal processing units be appropriately set in consideration of a manufacturing cost of the blood flow measuring device 100, a design size of the blood flow measuring device 100 and the like.

The light source unit 10 irradiates a living body (not illustrated) with coherent light. Note that, the light applied by the light source unit 10 does not have to be complete coherent light, and may be irradiation light close to the coherent light, for example, laser light and the like.

Each of a plurality of light reception units (20-1, 20-2, 20-3) receives reflected light of the coherent light and converts intensity of the reflected light into a current signal. For example, photodiodes may be used as the light reception units (20-1, 20-2, 20-3).

A frequency of scattered light scattered by stationary living tissue out of the reflected light generated by irradiation of the coherent light is the same as a frequency of the applied coherent light. In contrast, a frequency of scattered light scattered by scattering substances (mainly red blood cells) moving in the blood vessel of the skin is slightly subjected to the Doppler shift. Interference light generated by interference of these two scattered lights is detected by each of a plurality of light reception units (20-1, 20-2, 20-3). Then, each of a plurality of signal processing units (30-1, 30-2, 30-3) performs frequency analysis processing on an optical beat of the interference light, so that a blood flow velocity is calculated.

(2) Signal Processing Unit

Each of a plurality of signal processing units (30-1, 30-2, 30-3) converts the current signal into a voltage signal. Each of a plurality of signal processing units (30-1, 30-2, 30-3) may include a smoothing unit. FIG. 2 is a configuration diagram of one embodiment of the blood flow measuring device including the smoothing unit. As illustrated in FIG. 2, a plurality of signal processing units (30-1, 30-2, 30-3) may at least include smoothing units (34-1, 34-2, 34-3), respectively. The smoothing units (34-1, 34-2, 34-3) are described later in detail.

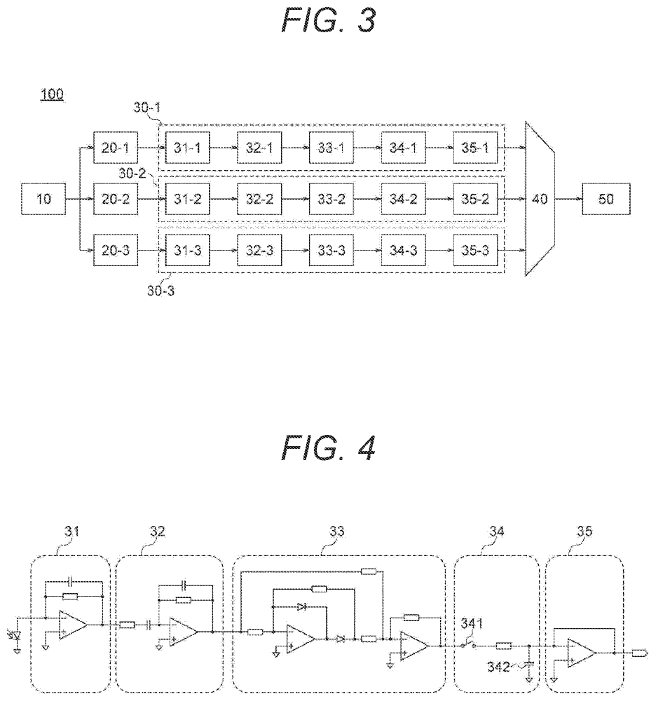

A configuration of each of a plurality of signal processing units (30-1, 30-2, 30-3) is not especially limited, but this may take an embodiment as illustrated in a configuration diagram in FIG. 3, for example. As illustrated in FIG. 3, the signal processing unit 30-1 may further include an IV conversion unit 31-1, a noise removal/differentiation unit 32-1, a rectification unit 33-1, and a buffer 35-1. The signal processing unit 30-2 and the signal processing unit 30-3 may also take the configuration similar to that of the signal processing unit 30-1.

The IV conversion unit 31-1 converts the current signal output from the light reception unit 20-1 into the voltage signal. For example, an operational amplifier and the like may be used as the IV conversion unit 31-1.

The noise removal/differentiation unit 32-1 first removes high-frequency noise. For example, a low-pass filter and the like may be used as the noise removal/differentiation unit 32-1. Moreover, the noise removal/differentiation unit 32-1 differentiates the voltage signal from which the noise is removed.

The rectification unit 33-1 rectifies a frequency characteristic of the voltage signal obtained by the noise removal/differentiation unit 32-1. The rectification may be full-wave rectification or half-wave rectification. In the full-wave rectification, a negative voltage of the voltage signal input to the rectification unit 33-1 is converted/rectified into a positive voltage. In the half-wave rectification, the negative voltage of the voltage signal input to the rectification unit 33-1 is erased.

The smoothing unit 34-1 smooths variation in voltage of the voltage signal rectified by the rectification unit 33-1.

The buffer 35-1 converts impedance. In order for each of a plurality of signal processing units (30-1, 30-2, 30-3) to output the voltage signal as accurately as possible, it is desirable that input impedance be as large as possible and output impedance be as small as possible. The buffer 35-1 may reduce the output impedance. For example, a source follower and the like may be used as the buffer 35-1.

Note that, FIG. 4 is a circuit diagram illustrating one embodiment of the signal processing unit 30-1 for reference. As illustrated in FIG. 4, the signal processing unit 30-1 may include an IV conversion unit 31, a noise removal/differentiation unit 32, a rectification unit 33, a smoothing unit 34, and a buffer 35. Note that, a configuration with which the signal processing unit 30-1 is realized is not limited to the circuit diagram in FIG. 4. Furthermore, although the circuit diagram in FIG. 4 is a single-ended system, this may also be a differential transmission system.

(3) Multiplexing Unit

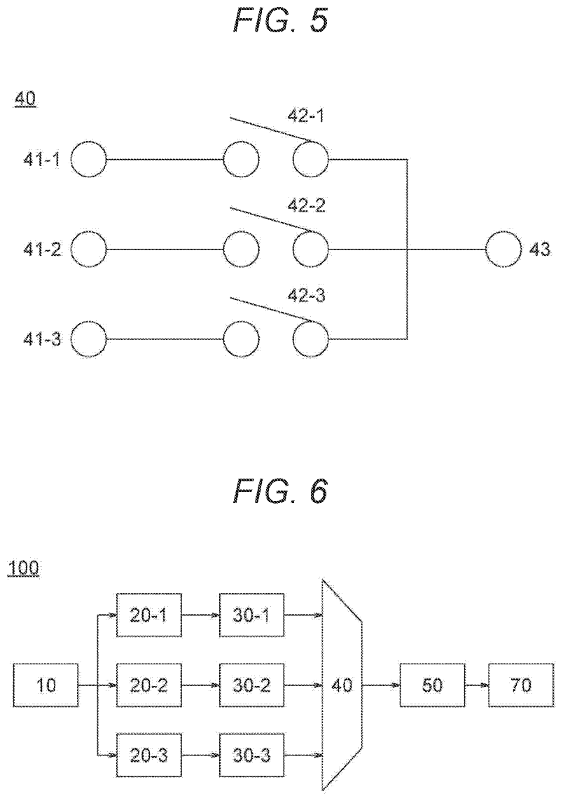

It is described with reference to FIG. 3 again. The multiplexing unit 40 multiplexes a plurality of the voltage signals into at least one multiplexed voltage signal. An analog switch, a multiplexer and the like may be used as the multiplexing unit 40, for example. FIG. 5 is a configuration diagram of one embodiment of the multiplexing unit 40.

As illustrated in FIG. 5, the multiplexing unit 40 includes a plurality of input terminals (41-1, 41-2, 41-3), a plurality of switches (42-1, 42-2, 42-3), and at least one output terminal 43. A plurality of input terminals (41-1, 41-2, 41-3) is connected to a plurality of switches (42-1, 42-2, 42-3), respectively. Each of a plurality of switches (42-1, 42-2, 42-3) is connected to the output terminal 43.

The multiplexing unit 40 sequentially switches the voltage signal to be input by turning on/off a plurality of switches (42-1, 42-2, 42-3). As a result, the multiplexing unit 40 may multiplex a plurality of voltage signals into at least one multiplexed voltage signal.

It is described with reference to FIG. 3 again. The AD conversion unit 50 samples the multiplexed voltage signal multiplexed by the multiplexing unit 40 at a predetermined cycle of sampling timings to obtain a multiplexed digital signal. For example, an AD converter may be used as the AD conversion unit 50.

(4) Averaging Processing Unit

Noise might occur due to a short cycle of the sampling timings and the like. In order to reduce the noise contained in the multiplexed digital signal, the blood flow measuring device 100 may further be provided with an averaging processing unit 70. FIG. 6 is a configuration diagram of one embodiment of the blood flow measuring device 100. As illustrated in FIG. 6, the blood flow measuring device 100 may further be provided with the averaging processing unit 70.

The averaging processing unit 70 performs averaging processing on the multiplexed digital signal. As a result, the averaging processing unit 70 may obtain a low-noise signal. For example, a microcomputer and the like may be used as the averaging processing unit 70.

The averaging processing is intended to mean, for example, dividing the sum of values of a plurality of voltage signals by the number of the voltage signals.

In this manner, the multiplexing unit 40 multiplexes a plurality of voltage signals into at least one multiplexed voltage signal, so that at least one subsequent AD conversion unit 50 is sufficient. With this configuration, one AD conversion unit 50 may process a plurality of voltage signals.

However, since the multiplexing unit 40 sequentially switches the voltage signal to be input, the sampling timing of the AD conversion unit 50 is deviated for each voltage signal. When it is described with reference to FIG. 5, for example, when the voltage signals having the same waveform are simultaneously input to the input terminals (41-1, 41-2, 41-3), respectively, the multiplexing unit 40 sequentially switches the voltage signal to be input, so that a timing at which a first switch 42-1 is turned on and a timing at which a second switch 42-2 is turned on are different from each other. Therefore, it becomes difficult to obtain a plurality of voltage signals at the same time. As a result, the averaging processing at the same time by the averaging processing unit 70 becomes difficult.

(5) Smoothing Unit

Therefore, as illustrated in FIG. 4, the smoothing unit 34 may include a charging switch 341 and a capacitor 342. As a result, an output timing of the voltage signal output by the signal processing unit 30 may be controlled.

Since the charging switch 341 alternately repeats turning on/off, the capacitor 342 alternately repeats a state of being charged with an electric charge regarding the voltage signal and a state of discharging the electric charge. As a result, the smoothing unit 34 samples the electric charge.

More specifically described, in a case where it is not the output timing of this voltage signal, the charging switch 341 is turned on and the capacitor 342 is charged with the electric charge regarding this voltage signal. In a case of the output timing of this voltage signal, the charging switch 341 is turned off and the electric charge with which the capacitor 342 is charged is discharged. As a result, the smoothing unit 34 may output the voltage signal at an appropriate timing.

Furthermore, the capacitor 342 alternately repeats the charge and discharge of the electric charge, so that the voltage signal is converted into a flat and smooth voltage signal. As a result, the smoothing unit 34 may obtain an effective value that correlates with the blood flow velocity.

By the way, in a case where a time for the capacitor 342 to be charged with the electric charge is short, the value of the voltage signal is not constant and becomes noise. Therefore, it is desirable that the time for the capacitor 342 to be charged with the electric charge is as long as possible.

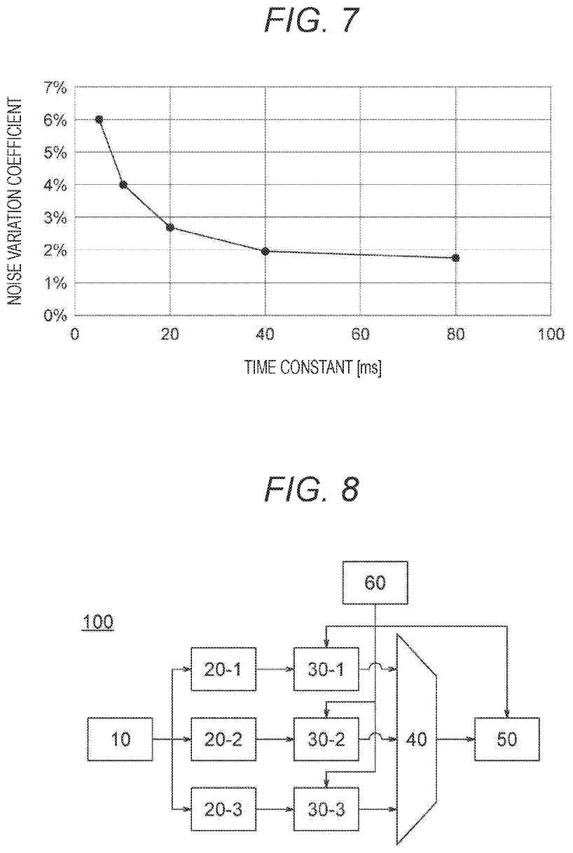

Here, FIG. 7 illustrates a simulation result of a time constant of the smoothing unit 34 and a noise variation coefficient of the voltage signal. The simulation result illustrated in FIG. 7 is obtained by simulating the noise variation coefficient of the voltage signal by changing the time constant when the cycle of the sampling timings of the AD conversion unit 50 is 20 milliseconds. Note that, the noise variation coefficient indicates an amount of noise contained in the voltage signal. The noise variation coefficient may be calculated by dividing amplitude of the noise signal by an average value of the voltage signals.

As illustrated in FIG. 7, the longer the time constant, the smaller the noise variation coefficient of the voltage signal. In general, when the time for charging the capacitor with the electric charge is short, the value of the voltage signal is not constant, and the amount of noise contained in the voltage signal tends to increase. The noise amount may be reduced by lengthening the time constant. However, in general, as the time constant becomes longer, the waveform of the voltage signal output by the smoothing unit 34 tends to be blunted.

Therefore, as illustrated in FIG. 7, the time constant is desirably set to about 10 to 40 milliseconds. Moreover, the time constant is desirably set to about 20 milliseconds.

Note that, the time when it is desirable that the time constant be set to about 20 milliseconds is when the cycle of the sampling timings of the AD conversion unit 50 is 20 milliseconds. For example, when the cycle of the sampling timings of the AD conversion unit 50 is 50 milliseconds, the time constant is desirably set to about 50 milliseconds.

(6) Timing Control Unit

Since the averaging processing is required to be performed on the voltage signals at the same time, the timings at which a plurality of smoothing units (34-1, 34-2, 34-3) discharge the electric charge are desirably synchronized. Therefore, the blood flow measuring device 100 may be provided with a timing control unit that synchronizes the discharge timings. FIG. 8 is a configuration diagram of one embodiment of the blood flow measuring device 100 provided with the timing control unit.

As illustrated in FIG. 8, the blood flow measuring device 100 may further be provided with a timing control unit 60. The timing control unit 60 is connected to the AD conversion unit 50. More specifically described, the AD conversion unit 50 includes a plurality of sampling switches (not illustrated). The sampling switches are connected to the timing control unit 60.

Furthermore, the timing control unit 60 is connected to each of a plurality of signal processing units (30-1, 30-2, 30-3). More specifically described, each of a plurality of signal processing units (30-1, 30-2, 30-3) includes the smoothing unit (not illustrated). The smoothing unit includes the charging switch (not illustrated) and the capacitor (not illustrated). The charging switch is connected to the timing control unit 60.

The timing control unit 60 may synchronize the discharge timing with the sampling timing of the AD conversion unit 50. More specifically described, the timing control unit 60 may control turning on/off of the sampling switch of the AD conversion unit 50.

Moreover, the timing control unit 60 may synchronize the discharge timings of a plurality of signal processing units (30-1, 30-2, 30-3). More specifically described, the timing control unit 60 may control turning on/off of the charging switch of each of a plurality of signal processing units (30-1, 30-2, 30-3). For example, a relay and the like may be used as the timing control unit 60.

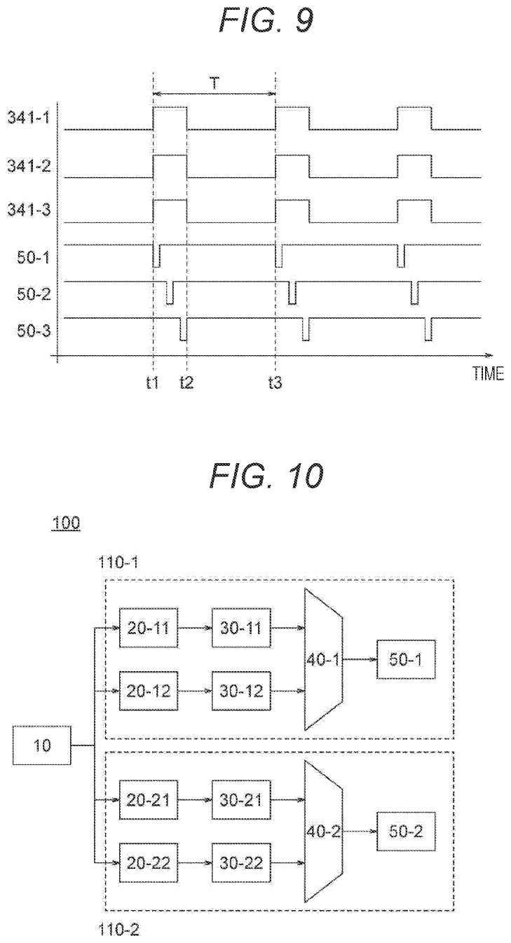

Subsequently, the sampling timing is described. The smoothing unit 34-1 is provided with a charging switch 341-1, the smoothing unit 34-2 is provided with a charging switch 341-2, and the smoothing unit 34-3 is provided with a charging switch 341-3. FIG. 9 is a timing chart of timings at which a plurality of charging switches (341-1, 41-2, 41-3) is turned on or off and the sampling timings of the AD conversion unit 50.

In FIGS. 9, 341-1, 41-2, and 41-3 represent timings at which the charging switches (341-1, 41-2, 41-3) are turned on or off, respectively. When a value is low, it is in an on state, that is, each of the charging switches (341-1, 41-2, 41-3) is closed. When the value is high, it is in an off state, that is, each of the charging switches (341-1, 41-2, 41-3) is opened.

50-1, 50-2, and 50-3 represent timings at which a plurality of sampling switches (50-1, 50-2, 50-3) of the AD conversion unit 50 is turned on or off, respectively. When a value is low, it is in an on state, that is, each of the sampling switches (50-1, 50-2, 50-3) is closed. When the value is high, it is in an off state, that is, each of the sampling switches (50-1, 50-2, 50-3) is opened.

When a first charging switch 341-1 is in the on state, a first capacitor (not illustrated) connected to the first charging switch 341-1 is charged with the electric charge. When the first charging switch 341-1 is in the off state, the electric charge with which the capacitor connected to the first charging switch 341-1 is charged is discharged.

When a second charging switch 341-2 is in the on state, a second capacitor (not illustrated) connected to the second charging switch 341-2 is charged with the electric charge. When the second charging switch 341-2 is in the off state, the electric charge with which the capacitor connected to the second charging switch 341-2 is charged is discharged.

When a third charging switch 341-3 is in the on state, a third capacitor (not illustrated) connected to the third charging switch 341-3 is charged with the electric charge. When the third charging switch 341-3 is in the off state, the electric charge with which the capacitor connected to the third charging switch 341-3 is charged is discharged.

The multiplexing unit 40 multiplexes the voltage signals regarding the discharged electric charges to obtain the multiplexed voltage signal. The AD conversion unit 50 samples the multiplexed voltage signal to obtain the multiplexed digital signal.

When a first sampling switch 50-1 is in the on state, the AD conversion unit 50 samples the electric charge of the first capacitor connected to the first charging switch 341-1. When a second sampling switch 50-2 is in the on state, the AD conversion unit 50 samples the electric charge of the second capacitor connected to the second charging switch 341-2. When a third sampling switch 50-3 is in the on state, the AD conversion unit 50 samples the electric charge of the third capacitor connected to the third charging switch 341-3.

A cycle T of each of the charging switches (341-1, 41-2, 41-3) is from time t1 to time t3.

Before time t1, the capacitor is charged with the electric charge. At time t1, each of the charging switches (341-1, 41-2, 41-3) changes from the on state to the off state. At that time, an amount of the electric charge with which the capacitor connected to each of the charging switches (341-1, 41-2, 41-3) is charged is fixed. That is, the value of the voltage signal is fixed.

Next, at time t1, the AD conversion unit 50 samples the electric charge of the first capacitor connected to the charging switch 341-1 (50-1). Subsequently, the AD conversion unit 50 samples the electric charge of the second capacitor connected to the charging switch 341-2 (50-2). Subsequently, the AD conversion unit 50 samples the electric charge of the third capacitor connected to the charging switch 341-3 (50-3).

At time t2, each of the charging switches (341-1, 41-2, 41-3) changes from the off state to the on state. At that time, the capacitor begins to be charged with the electric charge.

Although the sampling timings of 50-1, 50-2, and 50-3 are different from one another, the electric charge with which the capacitor is charged is sampled, so that the voltage signals at the same time may be sampled.

Note that, in the averaging processing, it is desirable that nearly identical noise is contained in the voltage signal processed by each of the signal processing units (30-1, 30-2, 30-3). Therefore, it is desirable that the respective light reception units (20-1, 20-2, 20-3) are arranged in identical positions from the light source unit.

2. Second Embodiment according to Present Technology (Configuration of Plural Groups)

FIG. 10 is a configuration diagram of a blood flow measuring device according to a second embodiment of the present technology. As illustrated in FIG. 10, a blood flow measuring device 100 includes a first group 110-1 and a second group 110-2. Note that, the number of groups is not limited to two.

The first group 110-1 includes a first light reception unit 20-11, a second light reception unit 20-12, a first signal processing unit 30-11, a second signal processing unit 30-12, at least one first multiplexing unit 40-1, and at least one first AD conversion unit 50-1. Note that, the number of the light reception units and that of the signal processing units are not limited to two.

Similarly, the second group 110-2 includes a third light reception unit 20-21, a fourth light reception unit 20-22, a third signal processing unit 30-21, a fourth signal processing unit 30-22, a second multiplexing unit 40-2, and a second AD conversion unit 50-2.

The light source unit 10 irradiates a living body (not illustrated) with coherent light. Each of a plurality of light reception units (20-11, 20-12, 20-21, 20-22) receives reflected light of the coherent light and converts intensity of the reflected light into a current signal. Each of a plurality of signal processing units (30-11, 30-12, 30-21, 30-22) converts the current signal into a voltage signal.

The first group 110-1 multiplexes the voltage signal processed by the first light reception unit 20-11 and the first signal processing unit 30-11 and the voltage signal processed by the second light reception unit 20-12 and the second signal processing unit 30-12 by the first multiplexing unit 40-1 to obtain a first multiplexed voltage signal V1. The first AD conversion unit 50-1 samples the first multiplexed voltage signal V1 to obtain a first multiplexed digital signal. Moreover, a first averaging processing unit (not illustrated) may perform averaging processing on the first multiplexed digital signal.

Similarly, the second group 110-2 multiplexes the voltage signal processed by the third light reception unit 20-21 and the third signal processing unit 30-21 and the voltage signal processed by the fourth light reception unit 20-22 and the fourth signal processing unit 30-22 by the second multiplexing unit 40-2 to obtain a second multiplexed voltage signal V2. The second AD conversion unit 50-2 samples the second multiplexed voltage signal V2 to obtain a second multiplexed digital signal. Moreover, a second averaging processing unit (not illustrated) may perform averaging processing on the second multiplexed digital signal.

Note that, the first group 110-1 and the second group 110-2 may share one AD conversion unit. For example, in a case where the AD conversion unit is powerful, the number of groups is small or the like, a plurality of groups may share one AD conversion unit.

Furthermore, in a case where the voltage signal obtained by one group is used as a certain reference signal, this group may include one light reception unit and one signal processing unit.

A phase of a sampling timing of the first AD conversion unit 50-1 and a phase of a sampling timing of the second AD conversion unit 50-2 may be the same or different from each other.

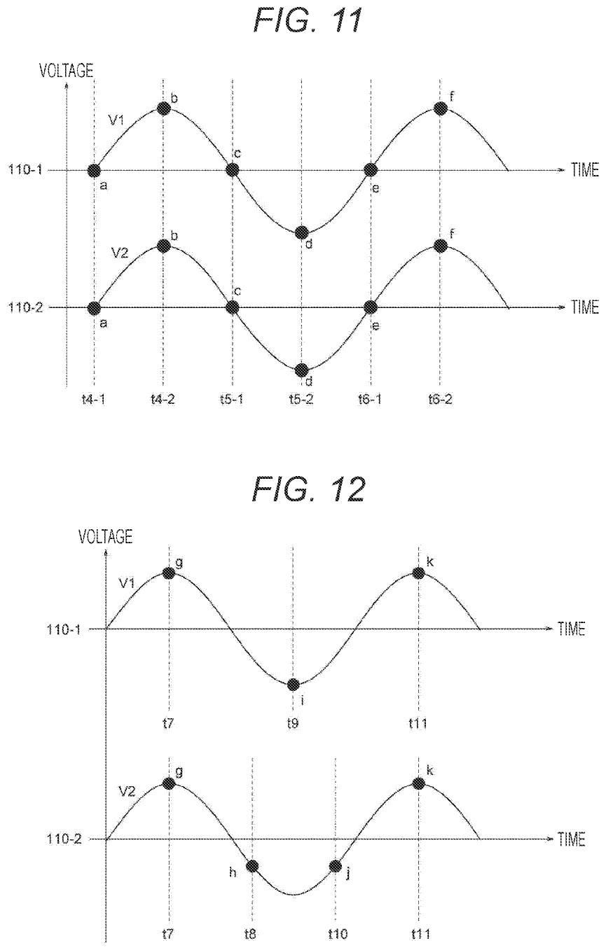

FIG. 11 is an illustrative view illustrating waveforms of the first multiplexed voltage signal V1 and the second multiplexed voltage signal V2. In FIG. 11, the waveform of the first multiplexed voltage signal V1 and the waveform of the second multiplexed voltage signal V2 are illustrated. Note that, the waveforms are simplified for simplifying the description.

The waveform of the first multiplexed voltage signal V1 and the waveform of the second multiplexed voltage signal V2 are the same. Therefore, information included in the first multiplexed voltage signal V1 and information included in the second multiplexed voltage signal V2 are the same.

Times t4-1, t5-1, and t6-1 are the sampling timings of the first AD conversion unit 50-1. Times t4-2, t5-2, and t6-2 are the sampling timings of the second AD conversion unit 50-2.

First, attention is focused on the first multiplexed voltage signal V1. At time t4-1, the first AD conversion unit 50-1 samples the voltage signal at point a. At time t5-1, the first AD conversion unit 50-1 samples the voltage signal at point c. At time t6-1, the first AD conversion unit 50-1 samples the voltage signal at point e.

Next, attention is focused on the second multiplexed voltage signal V2. At time t4-2, the second AD conversion unit 50-2 samples the voltage signal at point b. At time t5-2, the second AD conversion unit 50-2 samples the voltage signal at point d. At time t6-2, the second AD conversion unit 50-2 samples the voltage signal at point f.

In a case where the phase of the sampling timing of the first AD conversion unit 50-1 and the phase of the sampling timing of the second AD conversion unit 50-2 are the same, when describing with reference to FIG. 11, the first AD conversion unit 50-1 and the second AD conversion unit 50-2 may only sample the voltage signals at three points of a, c, and e points, or three points of b, d, and f points. However, in a case where the phases of the sampling timings are different from each other as illustrated in FIG. 11, the first AD conversion unit 50-1 and the second AD conversion unit 50-2 may sample the voltage signals at six points of a, b, c, d, e, and f points.

Therefore, when the case where the phase of the sampling timing of the first AD conversion unit 50-1 and the phase of the sampling timing of the second AD conversion unit 50-2 are the same is compared with the case where the phases are different from each other as illustrate in FIG. 11, the first AD conversion unit 50-1 and the second AD conversion unit 50-2 may sample twice the voltage signal data in the case illustrated in FIG. 11 although the cycles of the sampling timings of the AD conversion units are the same.

Therefore, the first AD conversion unit 50-1 and the second AD conversion unit 50-2 may more reliably sample the waveforms of the voltage signals. Therefore, the blood flow measuring device according to the present technology may more reliably perform the averaging processing on these voltage signals.

Alternatively, the cycle of the sampling timings of the first AD conversion unit 50-1 may differ from the cycle of the sampling timings of the second AD conversion unit 50-2. FIG. 12 is an illustrative view illustrating the waveforms of the first multiplexed voltage signal V1 and the second multiplexed voltage signal V2 in a case where the cycles of the sampling timings are different from each other. In FIG. 12, the waveform of the first multiplexed voltage signal V1 and the waveform of the second multiplexed voltage signal V2 are illustrated. The phase of the first multiplexed voltage signal V1 and the phase of the second multiplexed voltage signal V2 are the same.

Times t7, t9, and t11 are the sampling timings of the first AD conversion unit 50-1. Times t7, t8, t10, and t11 are the sampling timings of the second AD conversion unit 50-2.

First, attention is focused on the first multiplexed voltage signal V1. At time t7, the first AD conversion unit 50-1 samples the voltage signal at point g. At time t9, the first AD conversion unit 50-1 samples the voltage signal at point i. At time t11, the first AD conversion unit 50-1 samples the voltage signal at point k. Compared with the second multiplexed voltage signal V2, the cycle of the sampling timings of the first AD conversion unit 50-1 is longer than the cycle of the sampling timings of the second AD conversion unit 50-2.

Next, attention is focused on the second multiplexed voltage signal V2. At time t7, the second AD conversion unit 50-2 samples the voltage signal at point g. At time t8, the second AD conversion unit 50-2 samples the voltage signal at point h. At time t10, the second AD conversion unit 50-2 samples the voltage signal at point j. At time t11, the second AD conversion unit 50-2 samples the voltage signal at point k. Compared with the second multiplexed voltage signal V2, the cycle of the sampling timings of the second AD conversion unit 50-2 is shorter than the cycle of the sampling timings of the first AD conversion unit 50-1.

In the first multiplexed voltage signal V1 in which the cycle of the sampling timings is long, the voltage signal may be sampled at three points (points g, i, and k). In the second multiplexed voltage signal V2 in which the cycle of the sampling timings is short, the voltage signal may be sampled at four points (points g, h, i, and k).

The longer the cycle of the sampling timings, the larger an influence of a low blood flow velocity. By sampling the voltage signals at a plurality of sampling timings of different cycles of the sampling timings, it is possible to obtain a component at a high blood flow velocity and a component at a low blood flow velocity.

It is known that hardness of the blood vessel, that is, vascular stiffness may be obtained by using a ratio between the component of the high blood flow velocity and the component of the low blood flow velocity. When the blood vessel is soft, resistance is high and the blood flow velocity is low. When the blood vessels is hard, the resistance is low and the blood flow velocity is high.

As an example of an effect of obtaining the vascular stiffness, there may be prevention of arteriosclerosis. Arteriosclerosis results from a loss of physical flexibility of the arterial wall. Since arteriosclerosis is considered to cause stroke and cardiovascular diseases such as myocardial infarction responsible for high mortality, it may be said that it is of great significance to obtain the vascular stiffness.

3. Third Embodiment of Present Technology (Configuration in Two-Dimensional Direction)

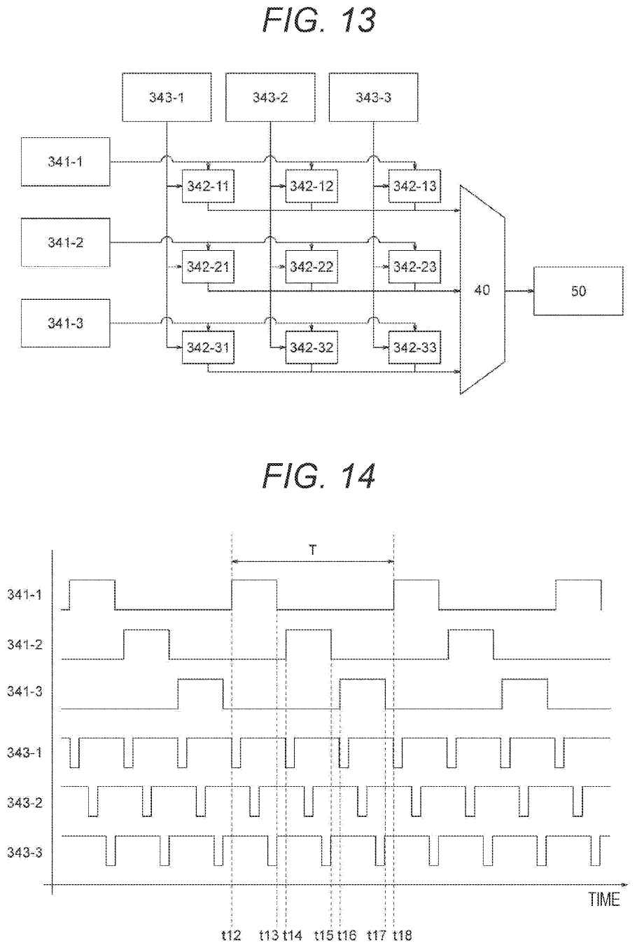

FIG. 13 is a configuration diagram of a blood flow measuring device according to a third embodiment of the present technology. As illustrated in FIG. 13, a smoothing unit is provided with a plurality of charging switches (first charging switch 341-1, second charging switch 341-2, third charging switch 341-3), a plurality of sampling switches (first sampling switch 343-1, second sampling switch 343-2, third sampling switch 343-3), and a plurality of capacitors (first capacitor 342-11, second capacitor 342-12, third capacitor 342-13, fourth capacitor 342-21, fifth capacitor 342-22, sixth capacitor 342-23, seventh capacitor 342-31, eighth capacitor 342-32, ninth capacitor 342-33).

Note that, the number of a plurality of charging switches (341-1, 41-2, 41-3) and that of a plurality of sampling switches (343-1 to 3) are not limited to three. Furthermore, the number of a plurality of capacitors (342-11 to 33) is not limited to nine.

The first charging switch 341-1 is connected to the first capacitor 342-11, the second capacitor 342-12, and the third capacitor 342-13. The second charging switch 341-2 is connected to the fourth capacitor 342-21, the fifth capacitor 342-22, and the sixth capacitor 342-23. The third charging switch 341-3 is connected to the seventh capacitor 342-31, the eighth capacitor 342-32, and the ninth capacitor 342-33.

The first sampling switch 343-1 is connected to the first capacitor 342-11, the fourth capacitor 342-21, and the seventh capacitor 342-31. The second sampling switch 343-2 is connected to the second capacitor 342-12, the fifth capacitor 342-22, and the eighth capacitor 342-32. The third sampling switch 343-3 is connected to the third capacitor 342-13, the sixth capacitor 342-23, and the ninth capacitor 342-33.

Each of a plurality of charging switches (341-1, 341-2, 341-3) alternately repeats turning on/off, so that each of a plurality of capacitors (342-11, 342-12, 342-13, 342-21, 342-22, 342-23, 342-31, 342-32, 342-33) alternately repeats a state of being charged with an electric charge regarding the voltage signal and a state of discharging the electric charge. Each of a plurality of sampling switches (343-1, 343-2, 343-3) alternately repeats turning on/off, so that the smoothing unit samples the electric charge.

More specifically described, in a case where it is not a sampling timing of the voltage signal regarding the first charging switch 341-1, the first charging switch 341 is turned on and the first capacitor 342-11, the second capacitor 342-12, and the third capacitor 342-13 are charged with the electric charge regarding this voltage signal. In a case of the sampling timing of this voltage signal, the first charging switch 341 is turned off and the electric charges with which the first capacitor 342-11, the second capacitor 342-12, and the third capacitor 342-13 are charged are discharged. Then, at the timings at which the first sampling switch 343-1, the second sampling switch 343-2, and the third sampling switch 343-3 are turned on, the smoothing unit samples each of the electric charges.

In parallel to charge or discharge of the first capacitor 342-11, the second capacitor 342-12, and the third capacitor 342-13, the other capacitors may be charged or discharged.

FIG. 14 is a timing chart of timings at which a plurality of charging switches (341-1, 341-2, 341-3) is turned on or off and timings at which a plurality of sampling switches (343-1, 343-2, 343-3) is turned on or off.

In FIGS. 14, 341-1, 341-2, and 341-3 represent timings at which a plurality of charging switches (341-1, 341-2, 341-3) is turned on or off, respectively. When a value is low, it is in an on state, that is, each of a plurality of charging switches (341-1, 341-2, 341-3) is closed. When the value is high, it is in an off state, that is, each of a plurality of charging switches (341-1, 341-2, 341-3) is opened.

Timings at which a plurality of sampling switches (343-1, 343-2, 343-3) is turned on or off are represented by 343-1, 343-2, and 343-3, respectively. When a value is low, it is in an on state, that is, each of a plurality of charging switches (343-1, 343-2, 343-3) is closed. When the value is high, it is in an off state, that is, each of a plurality of charging switches (343-1, 343-2, 343-3) is opened.

A cycle T of the charging switch 341-1 is from time t12 to time t18.

Immediately before time t12, each of the capacitors (342-11, 342-12, 342-13, 342-21, 342-22, 342-23, 342-31, 342-32, 342-33) is charged with an electric charge. At time t12, the first charging switch 341-1 changes from the on state to the off state. At that time, an amount of the electric charge with which each of the first capacitor 342-11, the second capacitor 342-12, and the third capacitor 342-13 connected to the first charging switch 341-1 is charged is fixed. That is, the value of the voltage signal is fixed.

Next, at time t12, the first sampling switch 343-1 is turned on, and the electric charge with which the first capacitor 342-11 is charged is sampled. Subsequently, the second sampling switch 343-2 is turned on, and the electric charge with which the second capacitor 342-12 is charged is sampled. Subsequently, the third sampling switch 343-3 is turned on, and the electric charge with which the third capacitor 342-13 is charged is sampled.

Even while the electric charges with which the first capacitor 342-11, the second capacitor 342-12, and the third capacitor 342-13 are charged are sampled, the other capacitors continue to be charged with the electric charge.

At time t13, the first charging switch 341-1 changes from the off state to the on state. At that time, the first capacitor 342-11, the second capacitor 342-12, and the third capacitor 342-13 are started to be charged with the electric charge.

Subsequently, similarly, at time t14, the second charging switch 341-2 changes from the on state to the off state. At that time, an amount of the electric charge with which each of the fourth capacitor 342-21, the fifth capacitor 342-22, and the sixth capacitor 342-23 connected to the second charging switch 341-2 is charged is fixed. That is, the value of the voltage signal is fixed.

Subsequently, similarly, at time t14, the first sampling switch 343-1 is turned on, and the electric charge with which the fourth capacitor 342-21 is charged is sampled. Subsequently, the second sampling switch 343-2 is turned on, and the electric charge with which the fifth capacitor 342-22 is charged is sampled. Subsequently, the third sampling switch 343-3 is turned on, and the electric charge with which the sixth capacitor 342-23 is charged is sampled.

At time t15, the second charging switch 341-2 changes from the off state to the on state. At that time, the fourth capacitor 342-21, the fifth capacitor 342-22, and the sixth capacitor 342-23 are started to be charged with the electric charge.

Subsequently, similarly, at time t16, the third charging switch 341-3 changes from the on state to the off state. At that time, an amount of the electric charge with which each of the seventh capacitor 342-31, the eighth capacitor 342-32, and the ninth capacitor 342-33 connected to the third charging switch 341-3 is charged is fixed. That is, the value of the voltage signal is fixed.

Subsequently, similarly, at time t16, the first sampling switch 343-1 is turned on, and the electric charge with which the seventh capacitor 342-31 is charged is sampled. Subsequently, the second sampling switch 343-2 is turned on, and the electric charge with which the eighth capacitor 342-32 is charged is sampled. Subsequently, the third sampling switch 343-3 is turned on, and the electric charge with which the ninth capacitor 342-33 is charged is sampled.

At time t17, the third charging switch 341-3 changes from the off state to the on state. At that time, the seventh capacitor 342-31, the eighth capacitor 342-32, and the ninth capacitor 342-33 are started to be charged with the electric charge.

In this manner, noise contained in the voltage signal may be reduced by lengthening the time for each of a plurality of capacitors (342-11 to 33) to be charged with the electric charge as long as possible.

The multiplexing unit 40 multiplexes a plurality of voltage signals corresponding to the electric charges into one multiplexed voltage signal. The AD conversion unit 50 samples this multiplexed voltage signal to obtain a multiplexed digital signal. The digital signal contains a plurality of pieces of voltage information that is multiplexed.

In order to reduce the noise contained in the voltage signal, the blood flow measuring device 100 may further be provided with an averaging processing unit (not illustrated). The averaging processing unit performs averaging processing on the multiplexed digital signal. As a result, the blood flow measuring device 100 may obtain a low-noise signal.

Note that, it is possible that the timings at which the respective charging switches (341-1, 341-2, 341-3) are turned on or off do not coincide with one another as illustrated in FIG. 14 or coincide with one another. In a case where the timings do not coincide with one another, as described above, the electric charges with which the three capacitors connected to the respective charging switches (341-1, 341-2, 341-3) are charged are subjected to the averaging processing. On the other hand, in a case where the timings coincide with one another, the electric charges with which the nine capacitors are charged are subjected to the averaging processing. Since there are more capacitors in the latter case than in the former case, more accurate blood flow velocity information may be obtained.

Note that arrangement of the light reception unit, the signal processing unit or the like is not especially limited. For example, by arranging the light reception units in a two-dimensional array manner as in an image sensor, blood flow velocity distribution may be obtained in a two-dimensional direction. By obtaining the same in the two-dimensional direction, for example, the blood flow velocity for each blood vessel may be measured.

Moreover, since the blood flow measuring device 100 is provided with a lens, the blood flow velocity distribution in a wide range may be obtained.

Conventionally, there has been a technology referred to as a laser Doppler imaging method. In this laser Doppler imaging method, a single light reception unit obtains a blood flow map in a two-dimensional region.

Compared with this method, the technology according to the third embodiment may obtain blood flow information in the two-dimensional region in a short time, so that blood flow distribution of a living body may be obtained at a high frame rate. Therefore, for example, a massage effect may be quantified. This is because a blood flow in a site where the massage effect is high becomes faster.

Note that, the effect described in this specification is illustrative only; the effect is not limited thereto and there may also be another effect.

Note that, the present technology may also have a following configuration.

[1] A blood flow measuring device provided with:

at least one light source unit;

a plurality of light reception units;

a plurality of signal processing units;

at least one multiplexing unit; and

at least one AD conversion unit, in which

the light source unit irradiates a living body with coherent light,

each of the plurality of light reception units receives reflected light of the coherent light and converts intensity of the reflected light into a current signal,

each of the plurality of signal processing units converts the current signal into a voltage signal,

the multiplexing unit multiplexes a plurality of voltage signals into at least one multiplexed voltage signal, and

the AD conversion unit samples the multiplexed voltage signal to obtain a multiplexed digital signal.

[2] The blood flow measuring device according to [1], in which the signal processing unit includes a smoothing unit.

[3] The blood flow measuring device according to [2], in which

the smoothing unit includes

a charging switch, and

a capacitor,

the charging switch alternately repeats turning on and off, and

the capacitor alternately repeats a state of being charged with an electric charge regarding the voltage signal and a state of discharging the electric charge, so that the electric charge is sampled.

[4] The blood flow measuring device according to any one of [1] to [3], further provided with:

an averaging processing unit, in which

the averaging processing unit performs averaging processing on the multiplexed digital signal to obtain a low-noise signal.

[5] The blood flow measuring device according to [3], further provided with:

a timing control unit, in which

the timing control unit synchronizes a discharge timing of the capacitor with a sampling timing of the AD conversion unit.

[6] The blood flow measuring device according to [5], in which the timing control unit synchronizes discharge timings of a plurality of capacitors.

[7] The blood flow measuring device according to [5] or [6], in which phases of a plurality of sampling timings are different from each other.

[8] The blood flow measuring device according to any one of [5] to [7], in which cycles of a plurality of sampling timings are different from each other.

[9] The blood flow measuring device according to any one of [2] to [8], in which

the smoothing unit is provided with

a plurality of charging switches,

a plurality of sampling switches, and

a plurality of capacitors, and

at a timing at which each of the plurality of charging switches is turned on or off, an electric charge regarding the voltage signal with which each of the plurality of capacitors is charged is discharged and each of the plurality of sampling switches is turned on or off, so that the electric charge is sampled.

### REFERENCE SIGNS LIST

100 Blood flow measuring device
10 Light source unit
20 Light reception unit
30 Signal processing unit
31 IV conversion unit
32 Noise removal/differentiation unit
33 Rectification unit
34 Smoothing unit
341 Charging switch
342 Capacitor
343 Sampling switch
35 Buffer
40 Multiplexing unit
50 AD conversion unit
60 Timing control unit
70 Averaging processing unit
110-1 First group
110-2 Second group
V1 First multiplexed voltage signal
V2 Second multiplexed voltage signal

1. A blood flow measuring device comprising:
at least one light source unit;
a plurality of light reception units;
a plurality of signal processing units;
at least one multiplexing unit; and
at least one AD conversion unit, wherein
the light source unit irradiates a living body with coherent light,
each of the plurality of light reception units receives reflected light of the coherent light and converts intensity of the reflected light into a current signal,
each of the plurality of signal processing units converts the current signal into a voltage signal,
the multiplexing unit multiplexes a plurality of voltage signals into at least one multiplexed voltage signal, and
the AD conversion unit samples the multiplexed voltage signal to obtain a multiplexed digital signal.

2. The blood flow measuring device according to claim 1, wherein
the signal processing unit includes a smoothing unit.

3. The blood flow measuring device according to claim 2, wherein
the smoothing unit includes
a charging switch, and
a capacitor,
the charging switch alternately repeats turning on and off, and
the capacitor alternately repeats a state of being charged with an electric charge regarding the voltage signal and a state of discharging the electric charge, so that the electric charge is sampled.

4. The blood flow measuring device according to claim 1, further comprising:
an averaging processing unit, wherein
the averaging processing unit performs averaging processing on the multiplexed digital signal to obtain a low-noise signal.

5. The blood flow measuring device according to claim 3, further comprising:
a timing control unit, wherein
the timing control unit synchronizes a discharge timing of the capacitor with a sampling timing of the AD conversion unit.

6. The blood flow measuring device according to claim 5, wherein
the timing control unit synchronizes discharge timings of a plurality of capacitors.

7. The blood flow measuring device according to claim 5, wherein
phases of a plurality of sampling timings are different from each other.

8. The blood flow measuring device according to claim 5, wherein
cycles of a plurality of sampling timings are different from each other.

9. The blood flow measuring device according to claim 2, wherein
the smoothing unit is provided with
a plurality of charging switches,
a plurality of sampling switches, and
a plurality of capacitors, and
at a timing at which each of the plurality of charging switches is turned on or off, an electric charge regarding the voltage signal with which each of the plurality of capacitors is charged is discharged and each of the plurality of sampling switches is turned on or off, so that the electric charge is sampled.
